(19) United States
(12) Patent Application Publication
(10) Pub. No.: US 2021/0304887 A1
(43) Pub. Date: Sep. 30, 2021

(54) SYSTEM AND METHOD FOR FACILITATING DYNAMIC BIOMETRIC MEASUREMENT

(71) Applicant: CIPHER SKIN, Denver, CO (US)

(72) Inventors: Phillip Bogdanovich, Denver, CO (US); James Heathers, Somerville, MA (US); Timothy Gouin, Denver, CO (US); Guido Gioberto, Denver, CO (US); Shaka Bahadu, Emeryville, CA (US); Craig Weller, Evergreen, CO (US)

(73) Assignee: CIPHER SKIN, Denver, CO (US)

(21) Appl. No.: 17/212,374

(22) Filed: Mar. 25, 2021

Related U.S. Application Data
(60) Provisional application No. 63/000,859, filed on Mar. 27, 2020.

(51) Int. Cl. G16H 40/63

(52) U.S. Cl. CPC G16H 40/63

(57) ABSTRACT

A system and method for facilitating dynamic biometric measurement is disclosed. In particular, the system may include a garment to be worn on the body of the user. The garment may include a flexible circuit upon which a plurality of sensor arrays and a processing unit may be disposed upon. Once activated, the sensors of the plurality of sensor arrays may continuously measure sensor data associated with the user, such as when the user is at rest and when the user in motion. The sensors may transmit signals including the sensor data to the processing unit for aggregation, processing, and filtration. The processing unit may also analyze the sensor data to determine a physiological condition of the user, a health condition of the user, a state of the user, or a combination thereof. The analyzed sensor data may be transmitted to a device for presentation, further processing, and storage.

### FIELD OF THE INVENTION

The present application relates to wearable technologies, sensor technologies, biometric measurement technologies, health-related technologies, data aggregation and analysis technologies, and computing technologies, and more particularly, to a system and method for facilitating dynamic biometric measurement.

### BACKGROUND

In today's society, measuring biometric information for users to determine their physiological statuses and/or health statuses has become increasingly important. For example, the blood oxygen content of a user is a crucial clinical measure of particular relevance to cardiovascular, pulmonary, and lifestyle diseases, and its measurement is a core component of modern critical care. Blood oxygen content is almost always measured in peripheral tissue (often from the ear or finger), and in a clinical or laboratory environment (often a hospital or other critical care setting). Notably, dynamic oxygen measurement during motion, exercise, or activities of daily living is much less common, and is challenging for several reasons. For example, the amount of blood present in peripheral tissues changes (with temperature, exercise, age, tissue perfusion, etc.), which affects measurement accuracy, movement often destroys optical signals measured from the body, and monitoring from the fingertip interferes with the use of the hands. An alternative to monitoring peripheral blood oxygen is measuring arterial blood gas directly. This involves puncturing an artery, and is not possible outside surgical environments. That being said, blood oxygen monitoring outside a critical care setting and during regular human movement is necessary to monitor sudden changes in respiratory status and to model that status over time.

The use of an oximeter to determine blood oxygen saturation in peripheral tissues (SpO2) has been in clinical practice for around 40 years. An oximeter emits multiple wavelengths of light through tissue, which are differentially absorbed by oxygenated and deoxygenated blood, and the transmission or absorption of this light is measured by a sensor array which approximates blood oxygen saturation with a high degree of reliability and accuracy. This method has been used extensively in wrist, finger, and ear mounted devices. Usually SpO2 is deployed in a rigid housing or a flexible patch. Common physical embodiments include finger-based oxygen saturation sensors, small patches that are used on the fingers or feet of children, and purpose-built arrays that are used in laboratories. SpO2 is a field expedient replacement for the costly, time intensive, and painful Air Blood Gas (ABG) test. The test takes approximately 15 minutes to run and while more accurate/definitive at the time of draw is also intermittent by nature. SpO2 monitoring is particularly helpful when monitoring intervals need to be greater than every 15 minutes or where pain is a consideration as is the case with children. SpO2 technology is mature and monitors are commoditized to the point that they are small in form, can be purchased for less than a hundred dollars, and are highly accurate. Because SpO2 sensors use light to determine the amount of O2 in the blood stream, they also contain other signals implied by the pulsatile waveform produced by the heartbeat, and most SpO2 monitors on the market measure both blood O2 and heart rate. Other functions that can be extrapolated from accurate data include Perfusion Index (PI) and estimated Respiration Rate (RR). Research has also attempted to calculate further vascular factors from other features pulsatile waveform. The general durability of a device is also a major consideration. Current units are designed to be small, easy to implement, and reusable. More advanced SpO2 monitors may wirelessly connect to apps running on third party devices such as phones and can also be integrated into other medical equipment like ECG machines either by a wired or wireless methodology.

A major problem with using SpO2 while the body is in motion is the signal noise/artifact created during a user's movement, as the shifting of sensors relative to the underlying tissue disrupts the signal. In large part, these issues and many others can be specifically attributed to the size and spacing of sensor arrays utilized to produce light and capture its reflectance/absorbance. These limitations may directly related to the size of the primary implement or embodiment. For instance, the surface area available for light transmission and reception from an LED and sensor array on the bottom of a watch or inside of a finger clip. As a result, based on the foregoing, current technologies and processes may be modified and improved so as to provide enhanced functionality and features for users and systems to effectively and dynamically monitor biometric and/or other data associated with a user. Such enhancements and improvements may provide for improved user satisfaction, higher quality data, increased safety, increased reliability, increased accuracy, increased efficiencies, substantially reduced costs for businesses and individuals, and increased ease-of-use for users.

### SUMMARY

A system and methods for facilitating dynamic biometric measurement via a garment worn by a user are disclosed. In particular, the increased surface area available on the inside of a garment makes it possible to integrate much larger, flexible sensor arrays into SpO2 monitoring via oximetry instead of the traditional finger and/or ear route. The system and methods are able to utilize large sections of the lower body, torso, and arms as measurement beds. The space is also sufficient to monitor and combine signals from multiple sites simultaneously, thereby reducing signal noise and increasing accuracy. Lower motion artifact disturbance to ongoing monitoring may achieved by the system and methods by simultaneously monitoring multiple locations by localizing and/or distributing arrays of SpO2 sensors. An array of SpO2 sensors may comprise one or more sensors, without limitations. In certain embodiments, distributed sensor arrays integrated into garments can make use of the following: a central power source, or a distributed power source on every sensor array; even compression within the same array and between arrays, or variable compression within the same array and between arrays depending on the sensor or array site; a connection topology between sensors that can be centralized in a master processing unit, or distributed on every sensor array.

In certain embodiments, the distributed sensor array can include additional computational resources at every discrete node array, including but not limited to a processor, memory, wired or wireless interfaces, or both. The circuit the sensor array is mounted on might be rigid, flexible, or of any form, and may include other components mounted on the circuit. The connection between sensors and sensor arrays can be wired, wireless or both, without limitations. Integrating dynamic SpO2 into clothing including a sensor matrix may also mean that prolonged, continuous measurement is unobtrusive and comfortable for the wearer. A matrix, for example, could include one or many sensor arrays, where each sensor array might include one or more sub-sensors arrays. The device can either be standalone or incorporated with multiple other metrics to include but not limited to single or multi-lead ECG, ambient temp, motion, and spatial orientation.

The dynamic biometric monitoring provided by the system and methods enable the measurement of continuous real-time SpO2 and other biometrics throughout the day during all movements or activities. Some examples of some common activities might include: running, jogging, walking, jumping, squatting, biking, and/or other activities. Other examples of common movements in a physical therapy clinic might include: shoulder flexion, shoulder extension, shoulder adduction, shoulder abduction, shoulder rotation, elbow flexion, elbow extension, hip flexion, hip extension, hip adduction, hip abduction, hip rotation, knee flexion, knee extension. In certain embodiments, the system and methods may utilize photoplethysmography to monitor oxygen saturation in the blood and the architected solution of integrating SpO2 and other biometric capabilities into a flexible, wearable garment of any kind is novel. Further, the integration of dynamic SpO2 and other biometric monitoring capabilities into a garment facilitates effective signal noise processing, flexibility, and general circuit design.

To that end, in one embodiment according to the present disclosure, a system for facilitating dynamic biometric measurement via a garment is disclosed. In particular, the system may include a garment to be worn on a body of a user and a flexible circuit disposed on the garment. The system may also include one or more sensor arrays disposed on the flexible circuit. The system may further include a processing unit communicatively linked to the sensor array and configured to perform operations of the system. The operations may include receiving one or more signals including sensor data from any number of sensors of the sensor arrays. In certain embodiments, the sensor data may be generated by the sensors of the sensor arrays while the user is at rest, while the body of the user is in motion, or a combination thereof. The processing unit may also perform an operation that includes processing the signals to determine a physiological condition of the user, a health condition of the user, a state of the user, or a combination thereof. In certain embodiments, the processing unit and/or garment may perform an operation that includes transmitting the signal, the physiological condition of the user, the health condition of the user, the state of the user, or a combination thereof, to a device for further analysis, display, storage, or a combination thereof.

In another embodiment, a method for facilitating dynamic biometric measurement via a garment is disclosed. The method may include utilizing a memory that stores instructions, and a processor that executes the instructions to perform the various functions of the method. In particular, the method may include integrating a sensor array and a light emitting diode into a flexible circuit of a garment to be worn on a body of a user. The method may include activating the sensor array and the light emitting diode, and obtaining sensor data from the sensor array while the user is at rest, while the user is in motion, or a combination thereof. Additionally, the method may include receiving, at a processing unit of the garment, a signal including the sensor data from the sensor array. Furthermore, the method may include processing the signal to determine a physiological condition, a health condition, a state of the user, or a combination thereof.

In another embodiment, a non-transitory computer-readable device is disclosed, which comprises instructions, which when loaded and executed by a processor, cause the processor to perform operations comprising: activating a sensor array and a light emitting diode integrated into a flexible circuit of a garment to be worn on a body of a user; obtaining sensor data from the sensor array while the user is at rest, while the user is in motion, or a combination thereof; receiving a signal including the sensor data from the sensor array; and processing the signal to determine a physiological condition, a health condition, a state of the user, or a combination thereof.

These and other features of the systems and methods for facilitating dynamic biometric measurement via a garment are described in the following detailed description, drawings, and appended claims.

### BRIEF DESCRIPTION OF THE DRAWINGS

FIG. 1 is a schematic diagram of a system for facilitating dynamic biometric measurement via a garment according to an embodiment of the present disclosure.

FIG. 2 is a schematic diagram illustrating a multisensor array for use with a garment of the system of FIG. 1 according to an embodiment of the present disclosure.

FIG. 3 is a schematic diagram illustrating a signal sensor array for use with a garment of the system of FIG. 1 according to an embodiment of the present disclosure.

FIG. 4 is a schematic diagram illustrating a processing unit, a distributed sensor array and a plurality of local arrays for use with a garment of the system of FIG. 1 according to an embodiment of the present disclosure.

FIG. 5 is a schematic diagram of a garment that may be utilized to facilitate dynamic biometric measurement in the system of FIG. 1 according to an embodiment of the present disclosure.

FIG. 6 is a flow diagram illustrating a sample method for facilitating dynamic biometric measurement via a garment according to an embodiment of the present disclosure.

FIG. 7 is a schematic diagram of a machine in the form of a computer system within which a set of instructions, when executed, may cause the machine to perform any one or more of the methodologies or operations of the systems and methods for facilitating dynamic biometric measurement via a garment.

### DETAILED DESCRIPTION OF THE INVENTION

A system 100 and methods for facilitating dynamic biometric measurement is disclosed. In particular, the system and methods may include utilizing a garment to be worn on the body of the user. The garment may include a flexible circuit upon which a plurality of sensor arrays and a processing unit may be disposed upon. Once activated, the sensors of the plurality of sensor arrays may continuously measure sensor data associated with the user, such as when the user is at rest and when the user in motion. The sensors may transmit signals including the sensor data to the processing unit for aggregation, processing, and filtration. The processing unit may also analyze the sensor data to determine a physiological condition of the user, a health condition of the user, a state of the user, or a combination thereof. The analyzed sensor data may be transmitted to a device for presentation, further processing, and storage.

In certain embodiments, the system and methods incorporate dynamic biometric monitoring into a garment of some type. A garment may include but not be limited to leggings, a shirt, sleeves, a long sleeve shirt, or a belt. The larger surface area offered by the garment “substrate” may allow for more effective monitoring resulting from a larger array of light emitting diodes, a larger surface area for the incorporation of light sensors, and more surface area providing space for more arrays in total consisting of light emitting diodes and light sensors. Notably, the increase in surface area over current implementations allows for distributed light emitted diodes plus light sensor arrays that are more sensitive, more accurate, and flexible. Because the dynamic SpO2 and other biometric monitors are built directly into clothing, sensors can be worn for prolonged periods of time to achieve continuous monitoring even during exercise and prolonged physical activity. Monitoring for prolonged periods of time allows for insights not only during times of activity and times of rest, but also in the transition between a resting and active state. Such capabilities would be particularly useful for monitoring elderly patients that are bedridden for prolonged periods of time and also individuals who may be suffering from disorders difficult to diagnose such as reactive airway disease or activity induced asthma.

The dynamic SpO2 and biometric monitoring techniques provided by the system and methods allow for continuous monitoring even during physical activity and for prolonged periods of time. Dynamic biometric monitoring can also be integrated into related technologies as an additional available sub-sensor. This means that for the first time out of a laboratory dynamic SpO2 (and other biometric data) can not only be collected but collected in conjunction with, and synchronously to electrocardiogram data, motion data, temperature data, ambient temperature data, relative position in space data, muscle and tissue expansion data, and/or any other data.

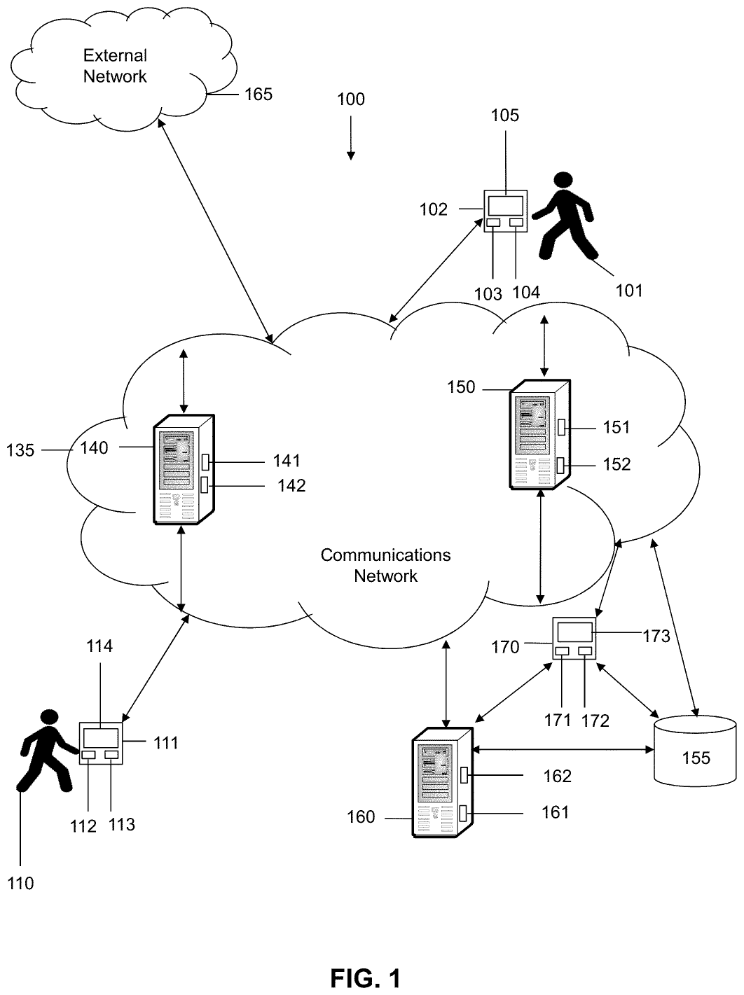

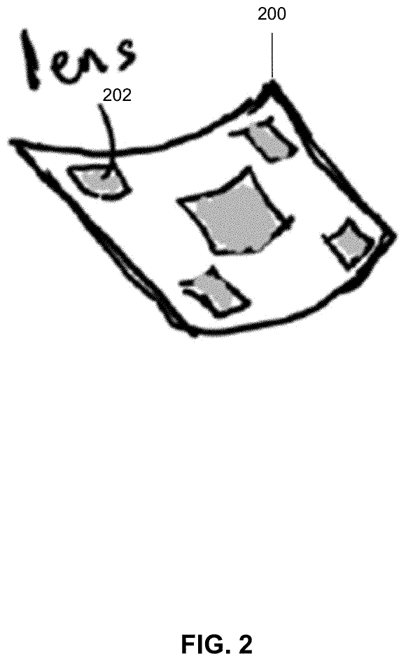

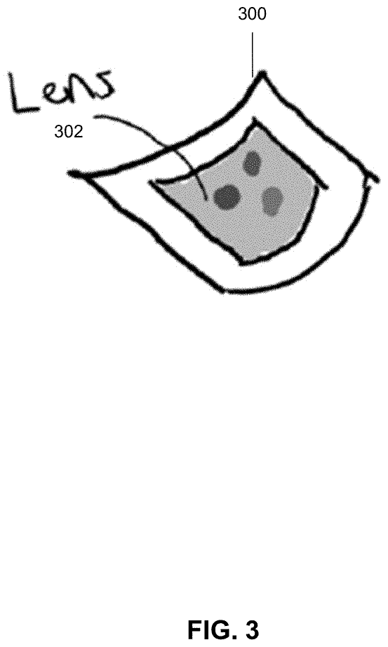

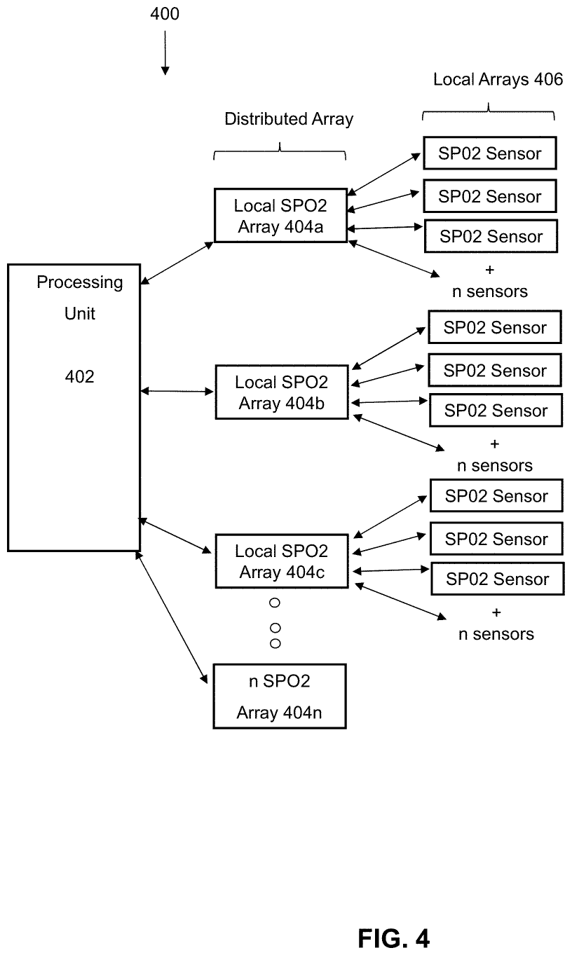

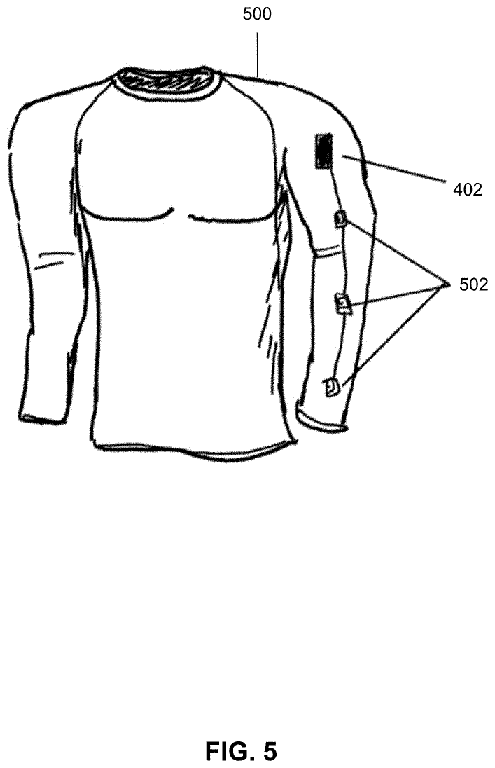

As shown in FIGS. 1-7, a system 100 and method for facilitating dynamic biometric measurement via a garment are disclosed. The system 100 may be configured to support, but is not limited to supporting, monitoring applications and services, sensor-based applications and services, biometric data analysis applications and services, wearable device applications and services, health monitoring applications and services, communication applications and services, alert applications and services, data and content services, data aggregation applications and services, big data technologies, health analysis technologies, data synthesis applications and services, data analysis applications and services, computing applications and services, cloud computing services, internet services, satellite services, telephone services, software as a service (SaaS) applications, mobile applications and services, and any other computing applications and services. The system may include a first user 101, who may utilize a first user device 102 to access data, content, and applications, or to perform a variety of other tasks and functions. As an example, the first user 101 may utilize first user device 102 to access an application (e.g. a browser or a mobile application) executing on the first user device 102 that may be utilized to access web pages, data, and content associated with the system 100. In certain embodiments, the first user 101 may be an individual that wants to monitor his own biometric measurements in order to determine his current state, health conditions, and/or physiological conditions.

The first user device 102 utilized by the first user 101 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, audio device interface, etc.) that may enable the first user 101 to interact with various applications executing on the first user device 102, to interact with various applications executing within the system 100, and to interact with the system 100 itself. In certain embodiments, the first user device 102 may be a computer, a laptop, a tablet device, a phablet, a server, a mobile device, a smartphone, a smart watch, and/or any other type of computing device. Illustratively, the first user device 102 is shown as a mobile device in FIG. 1. The first user device 102 may also include a global positioning system (GPS), which may include a GPS receiver and any other necessary components for enabling GPS functionality, accelerometers, gyroscopes, sensors, and any other componentry suitable for a mobile device. In certain embodiments, the first user device 102 may be configured to include any number of sensors, such as, but not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof.

In addition to the first user 101, the system 100 may include a second user 110, who may utilize a second user device 111 to access data, content, and applications, or to perform a variety of other tasks and functions. As with the first user 101, the second user 110 may be a user that is seeking to monitor his own biometric measurements to determine his health status, physiological status, and/or state. However, in certain embodiments, the second user 110 may be a supervisor of the first user 101, a physician, a first responder, an emergency personnel, a nurse, any type of health professional, any type of safety personnel, or any combination thereof. Much like the first user 101, the second user 110 may utilize second user device 111 to access an application (e.g. a browser or a mobile application) executing on the second user device 111 that may be utilized to access web pages, data, and content associated with the system 100. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. a screen, a monitor, a graphical user interface, etc.) that may enable the second user 110 to interact with various applications executing on the second user device 111, to interact with various applications executing in the system 100, and to interact with the system 100. In certain embodiments, the second user device 111 may be a computer, a laptop, a tablet device, a phablet, a server, a mobile device, a smartphone, a smart watch, and/or any other type of computing device. Illustratively, the second user device 111 may be a computing device in FIG. 1. The second user device 111 may also include any of the componentry described for first user device 102.

In certain embodiments, the first user device 102 and the second user device 111 may have any number of software applications and/or application services stored and/or accessible thereon. For example, the first and second user devices 102, 111 may include applications for analyzing biometric and/or sensor data, applications for determining and analyzing health conditions, applications for determining and analyzing the physiological status of a user, applications for generating alerts, applications for analyzing and interpreting sensor data, artificial intelligence applications, machine learning applications, big data applications, applications for analyzing data, applications for integrating data, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 101, 110 to readily interact with the software applications.

The software applications and services may also be utilized by the first and second users 101, 110 to interact with any device in the system 100, any network in the system 100, or any combination thereof. For example, the software applications executing on the first and second user devices 102, 111 may be applications for receiving data, applications for storing data, applications for determining health conditions, physiological conditions and/or states of a user, applications for determining how to respond to a health condition, physiological condition and/or state of a user, applications for determining a physiological status of a user, applications for determining how to respond to an environmental condition (e.g. an environmental condition that may affect the first user 101), applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first and second user devices 102, 111 may include associated telephone numbers, internet protocol addresses, device identities, or any other identifiers to uniquely identify the first and second user devices 102, 111 and/or the first and second users 101, 110. In certain embodiments, location information corresponding to the first and second user devices 102, 111 may be obtained based on the internet protocol addresses, by receiving a signal from the first and second user devices 102, 111, or based on profile information corresponding to the first and second user devices 102, 111.

The system 100 may also include a communications network 135. The communications network 135 of the system 100 may be configured to link each of the devices in the system 100 to one another and/or to the garment 500 worn by a user of the system 100. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry, and may be controlled by a service provider. The communications network 135 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. Illustratively, server 140 and server 150 are shown as being included within communications network 135.

Notably, the functionality of the system 100 and/or garment 500 may be supported and executed by using any combination of the servers 140, 150, and 160. The servers 140, and 150 may reside in communications network 135, however, in certain embodiments, the servers 140, 150 may reside outside communications network 135. The servers 140, and 150 may be utilized to perform the various operations and functions provided by the system 100, such as those requested by applications executing on the first and second user devices 102, 111. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 150 may include a memory 151 that includes instructions, and a processor 152 that executes the instructions from the memory 151 to perform the various operations that are performed by the server 150. In certain embodiments, the servers 140, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 150 may be communicatively linked to the communications network 135, any network, any device in the system 100, or any combination thereof.

The database 155 of the system 100 may be utilized to store and relay information that traverses the system 100, cache information and/or content that traverses the system 100, store data about each of the devices in the system 100, and perform any other typical functions of a database. In certain embodiments, the database 155 may store the output from any operation performed by the system 100, operations performed and/or outputted by the any components of the garment 500, operations performed and/or outputted by any component, program, process, device, network of the system 100 and/or any component of FIGS. 1-8, or any combination thereof. For example, the database 155 may store data from sensor arrays of the garment 500 and/or analyses conducted by the processing unit 402 of the garment 500. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or be connected to a processor and memory to perform the various operations associated with the database 155. In certain embodiments, the database 155 may be connected to the servers 140, 150, 160, the first user device 102, the second user device 111, the garment 500, any devices in the system 100, any other device, any network, or any combination thereof.

The database 155 may also store information obtained from the system 100, store information associated with the first and second users 101, 110, store location information for the first and second user devices 102, 111 and/or first and second users 101, 110, store user profiles associated with the first and second users 101, 110, store device profiles associated with any device in the system 100 (e.g. the garment 500), store communications traversing the system 100, store user preferences, store demographic information for the first and second users 101, 110, store information associated with any device or signal in the system 100, store information relating to usage of applications accessed by the first and second user devices 102, 111, store any information obtained from any of the networks in the system 100, store historical data associated with the first and second users 101, 110, store device characteristics, store information relating to any devices associated with the first and second users 101, 110, or any combination thereof. The database 155 may store algorithms for analyzing sensor data obtained from the sensor arrays of the garment 500, algorithms for determining events, such as health conditions and/or physiological status, algorithms for determining activities that the users are suited for, algorithms conducting artificial intelligence and/or machine learning, algorithms for comparing sensor data to baseline and/or threshold values, any other algorithms for performing any other calculations and/or operations in the system 100, or any combination thereof. The database 155 may also be configured to store information relating to detected events, actions to perform in response to the detected events, information indicating whether one or more of the actions have been performed, information indicating which environment associated with what types of events and how the events typically occur, any other information provided by the system 100 and/or method 600, or any combination thereof. In certain embodiments, the database 155 may be configured to store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

The system 100 may also include a software application, which may be configured to perform and support the operative functions of the system 100. In certain embodiments, the application may be a website, a mobile application, a software application, or a combination thereof, which may be made accessible to users utilizing one or more computing devices, such as first user device 102 and second user device 111. The application of the system 100 may be accessible via an internet connection established with a browser program executing on the first or second user devices 102, 111, a mobile application executing on the first or second user devices 102, 111, or through other suitable means. Additionally, the application may allow users and computing devices to create accounts with the application and sign-in to the created accounts with authenticating username and password log-in combinations. The application may include a custom graphical user interface that the first user 101 or second user 110 may interact with by utilizing a web browser executing on the first user device 102 or second user device 111. In certain embodiments, the software application may execute directly as an installed program on the first and/or second user devices 102, 111. In certain embodiments, some or all of the software application may execute on the garment 500.

The software application may include multiple programs and/or functions that execute within the software application and/or are accessible by the software application. For example, the software application may include an application that generates web content, pages, and/or data that may be accessible to the first and/or second user devices 102, 111, the garment 500, the database 155, the external network 165, any type of program, any device and/or component of the system 100, or any combination thereof. The application that generates web content and pages may be configured to generate a graphical user interface and/or other types of interfaces for the software application that are accessible and viewable by the first and second users 101, 110 when the software application is loaded and executed on the first and/or second computing devices 102, 111. The graphical user interface for the software application may display content associated with sensor data and/or biometric data measured by the garment 100 and/or processed by the processing unit 402, determined health conditions, physiological conditions and/or states of the user, any other type of information, or any combination thereof. Additionally, the graphical user interface may display functionality provided by the software application that enables the first and/or second user 101, 110 and/or the first user device and/or second user device 111 to input parameters and requirements for the various process conducted by the system 100.

The system 100 may also include an external network 165. The external network 165 of the system 100 may be configured to link each of the devices in the system 100 to one another. For example, the external network 165 may be utilized by the first user device 102, the second user device 111, and/or the garment 500 to connect with other devices within or outside communications network 135. Additionally, the external network 165 may be configured to transmit, generate, and receive any information and data traversing the system 100. In certain embodiments, the external network 165 may include any number of servers, databases, or other componentry, and may be controlled by a service provider. The external network 165 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. In certain embodiments, the external network 165 may be outside the system 100 and may be configured to perform various functionality provided by the system 100, such as if the system 100 is overloaded and/or needs additional processing resources. In certain embodiments, the external network 165 may be configured to perform some or all of the operations conducted by the garment 500.

In certain embodiments, the system 100 may include a multi-sensor array 200 that may be integrated into a flexible circuit that may be integrated into fabric of a garment 500. The multi-sensor array 200 may include sensor lenses 202, which may be configured to contact and conform to the skin of the user when the garment 500 is worn by the user. The sensor lenses 202 may have varying surface areas and may be configured to measure biometric data of the user wearing the garment 500. In certain embodiments, any number of sensor lenses 202 may be utilized, and the sensor arrays 200 may be located anywhere on the garment 500. The sensor lenses 202 may be arranged in any configuration and/or pattern on the flexible circuit. In certain embodiments, the multi-sensor array 200 may include any number of light emitting diodes, which may be configured to emit light through tissue of the body of the user when activated. Based on the transmission and/or absorption of the light in the tissue, the multi-sensor array 200 may be determine a blood oxygen saturation of the user wearing the garment 500 and/or other biometric, health, physiological, and/or state information. In certain embodiments, the multi-sensor array 200 may allow for increased lens quantity, arrangement density and/or surface area to collect more and better signals to be processed for a more reliable output. Additionally, localized sensor arrays 200 (also 300, 404a-n, 406, and 502) may be distributed in any fashion in the garment 500 as needed. The system 100 may also include a single sensor array 300, which may include a single lens 302 to measure biometric data of the user wearing the garment 500. In certain embodiments, the single lens 302 may have a large surface area and multiple light emitting diodes to facilitate the measurement of biometric data. The sensors of the sensor arrays may be configured to be made of a flexible material and may include any number of sensors. In certain embodiments, additional sub-sensors may be embedded within the sensor arrays and may be interchanged depending on the type of sensors that are desired. The sensors may include, but are not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof.

As indicated above, the system 100 may include a garment 500, which may be worn by a user. The garment 500 may cover the entire body of the user, any body part of the user, or specific limbs of the user. The garment 500 may be configured to include any number of flexible circuits integrated into the fabric of the garment 500. The garment may also include a processing unit 402, which may be configured to process signals including sensor data (and biometric information) received from sensors in the sensor arrays 200, 300, 404a-n, and 406. The processing unit 402 may aggregate the signals and may analyze the signals to determine a physiological condition of the user, a health condition of the user, a state of the user, and/or any other information associated with the user wearing the garment 500. The garment 500 may also include a power source, such as a battery or other power source, that may provide power to the sensor arrays 200, 300, 404a-n, 406, the processing unit 402, and/or to any other components of the garment 500. The garment 500 may include memories, additional processors, communication components, storage devices, and/or any other desired devices as well. In certain embodiments, the garment 500 may also include a transceiver. The transceiver may include any of the functionality of a traditional transceiver. The transceiver may be configured to transmit signals generated by the components of the garment 500 to other devices and networks of the system 100, receive signals generated by any of the devices of the system 100, receive signals generated by the components of the garment 500, or a combination thereof. In certain embodiments, the transceiver may enable the garment 500 to communicate information gathered by the components of the garment 500 to other devices of the system 100 for further processing. In certain embodiments, the transceiver may enable the garment 500 to communicatively couple to any device in the system 100, the communications network 135, the external network 165, or a combination thereof.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, integrating sensor arrays and light emitting diodes into a flexible circuit of a garment for monitoring biometric measurements of a user; providing a power source in the garment to provide power to the sensor array and light emitting diodes integrated into the garment; activating the sensors and/or light emitting diodes so as to generate and obtain the biometric measurements of the user; receiving signals from the sensors of the sensor arrays that include the biometric measurements of the user; processing the signals to determine a state of the user, a physiological condition of the user, a health condition of the user, or a combination thereof; transmitting the processed biometric/sensor data to a device for further analysis, display, and/or storage; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

In certain embodiments, the system 100 may also include a computing device 170. The computing device 170 may include one or more processors 172 that may be configured to process any of the various functions of the system 100. The processors 172 may be software, hardware, or a combination of hardware and software. Additionally, the computing device 170 may also include a memory 171, which stores instructions that the processors 172 may execute to perform various operations of the system 100. For example, the computing device 170 may assist in processing loads handled by the various devices in the system 100, such as, but not limited to, the wearable devices 300, 400. In certain embodiments, for example, the garment 500 may offload certain types of less critical operations to the computing device 170 so that the processing unit 402 and/or other components of the garment 500 can focus on more critical operations.

Although FIGS. 1-7 illustrates specific example configurations of the various components of the system 100, the system 100 may include any configuration of the components, which may include using a greater or lesser number of the components. For example, the system 100 is illustratively shown as including a first user device 102, a second user device 111, a database 125, a communications network 135, a server 140, a server 150, a server 160, a database 155, an external network 165, a multi-sensor array 200, a single sensor array 300, a processing unit 402, sensor arrays 404a-n, local sensor arrays 406, a garment 500, and sensor arrays 502. However, the system 100 may include multiple first user devices 102, multiple second user devices 111, multiple databases 125, multiple communications networks 135, multiple servers 140, multiple servers 150, multiple servers 160, multiple databases 155, multiple external networks 165, multiple multi-sensor arrays 200, multiple single sensor arrays 300, multiple processing units 402, any number of sensor arrays 404a-n, any number of local sensor arrays 406, multiple garments 500, and any number of sensor arrays 502, and/or any number of any of the other components inside or outside the system 100. Similarly, the system 100 may include any number of data sources, applications, systems, and/or programs. Furthermore, in certain embodiments, substantial portions of the functionality and operations of the system 100 may be performed by other networks and systems that may be connected to system 100.

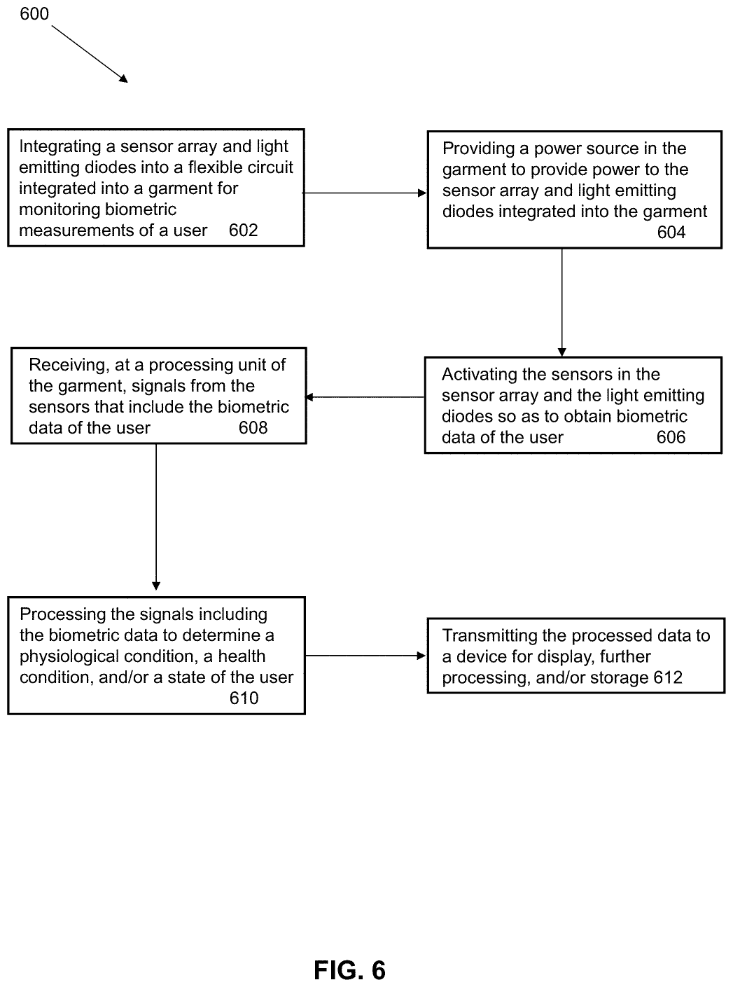

As shown in FIG. 6, an exemplary method 600 for facilitating dynamic biometric measurement via a garment is schematically illustrated. The method 600 may include, at step 602, integrating one or more sensor arrays and light emitting diodes into a flexible circuit of a garment to be worn by a user, such as first user 101. The sensor arrays and light emitting diodes may be utilized to measure biometric measurements of a user wearing the garment 500. In certain embodiments, the integrating may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof. At step 604, the method 600 may include providing a power source in the garment to provide power to the sensor arrays and light emitting diodes (and other components of the garment 500). In certain embodiments, the providing may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof.

At step 606, the method 600 may include activating the sensor arrays and the light emitting diodes so as to cause the sensor arrays and light emitting diodes to work together to measure biometric data associated with the user wearing the garment 500. In certain embodiments, the activating may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof. At step 608, the method 600 may include receiving, at a processing unit of the garment 500 (or other component of the system 100), signals from the sensors that include the measured biometric data of the user. In certain embodiments, the receiving may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof.

At step 610, the method 600 may include processing the signals including the biometric data to analyzing the data, filter the data, store the data, and/or perform any other operations on the data. In certain embodiments, the processing of the signals may be performed to determine a physiological condition and/or status of the user, a health condition and/or status of the user, and/or a state of the user. In certain embodiments, the processing may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof. At step 612, the method 600 may include transmitting the processed data to a device for display, further processing, storage, and/or other operations. In certain embodiments, the transmitting may be performed and/or facilitated by utilizing the first user device 102, the second user device 111, the server 140, the server 150, the server 160, the communications network 135, the external network 165, the database 155, any component of the garment 500, the computing device 170, any appropriate program, device, network, user, and/or process of the system 100, or a combination thereof. Notably, the method 600 may further incorporate any of the features and functionality described for the system 100 or as otherwise described herein.

The systems and methods disclosed herein may include additional functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and method 600. For example, through the use of the artificial intelligence and machine learning in conjunction with the system 100 and/or method 400, a reduced amount of computer operations need to be performed by the devices in the system 100 using the processors and memories of the system 100 than in systems that are not capable of machine learning as described in this disclosure. As an illustration and in certain embodiments, the system 100 may learn over time that biometric and sensor data are associated with certain physiological conditions, health conditions, and/or states of a user. For example, if the system 100 initially determines during a first occasion that measured biometric data of the user and/or is associated with a certain physiological condition, knowing this information, the system 100 may automatically determine the physiological condition for the user during a future second occasion if newly measured biometric and/or sensor data matches the biometric and/or sensor data from the first occasion. In such a context, less processing power needs to be utilized because the processors and memories do not need perform analyses and operations that have already been learned by the system 100. As a result, there are substantial savings in the usage of computer resources by utilizing the software, functionality, and algorithms provided in the present disclosure.

Notably, in certain embodiments, various functions and features of the system 100 and methods may operate without human intervention and may be conducted entirely by computing devices, robots, and/or processes. For example, in certain embodiments, multiple computing devices may interact with devices of the system 100 to provide the functionality supported by the system 100. Additionally, in certain embodiments, the computing devices of the system 100 may operate continuously to reduce the possibility of errors being introduced into the system 100. In certain embodiments, the system 100 and methods may also provide effective computing resource management by utilizing the features and functions described in the present disclosure. For example, in certain embodiments, while utilizing the sensors of the sensor arrays to measure sensor data, any selected device in the system 100 may transmit a signal to a computing device receiving or processing the sensor data that only a specific quantity of computer processor resources (e.g. processor clock cycles, processor speed, processor cache, etc.) may be dedicated to processing the sensor data, processing any other operation conducted by the system 100, or any combination thereof. For example, the signal may indicate an amount of processor cycles of a processor that may be utilized to process the sensor data and/or specify a selected amount of processing power that may be dedicated to processing the data and/or any of the operations performed by the system 100. In certain embodiments, a signal indicating the specific amount of computer processor resources or computer memory resources to be utilized for performing an operation of the system 100 may be transmitted from the first and/or second user devices 102, 111 and/or garment 500 to the various components and devices of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected, predetermined, and/or threshold value. In certain embodiments, the system 100 and methods may include transmitting signals to the memory devices utilized in the system 100, which indicate which specific portions (e.g. memory sectors, etc.) of the memory should be utilized to store any of the data utilized or generated by the system 100. Notably, the signals transmitted to the processors and memories may be utilized to optimize the usage of computing resources while executing the operations conducted by the system 100. As a result, such features provide substantial operational efficiencies and improvements over existing technologies.

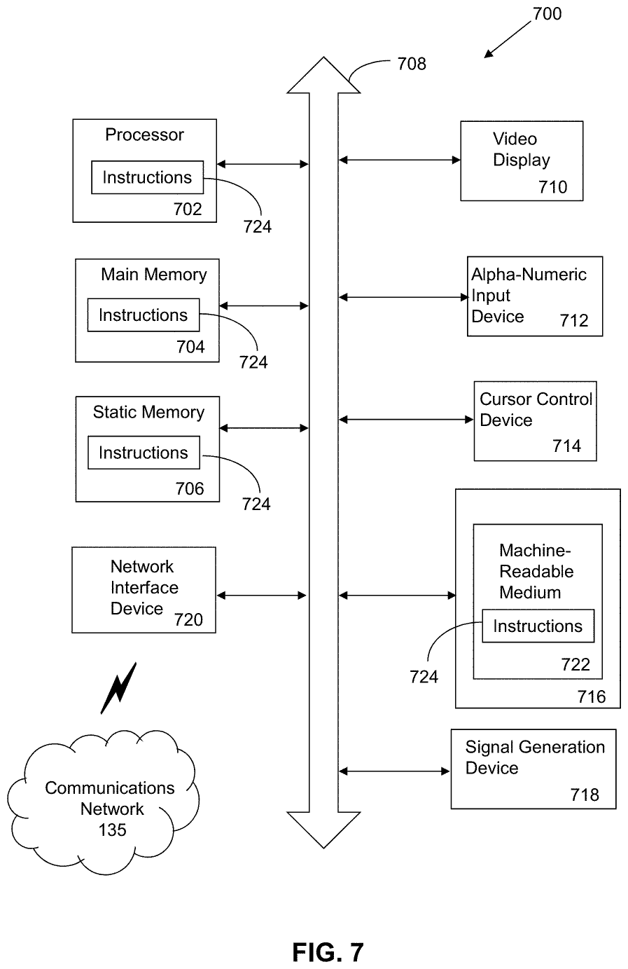

Referring now also to FIG. 7, at least a portion of the methodologies and techniques described with respect to the exemplary embodiments of the system 100 can incorporate a machine, such as, but not limited to, computer system 700, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100.

In some embodiments, the machine may operate as a standalone device. In some embodiments, the machine may be connected (e.g., using communications network 135, another network, or a combination thereof) to and assist with operations performed by other machines, programs, functions, and systems, such as, but not limited to, the first user device 102, the second user device 111, the server 140, the server 150, the database 155, the server 160, the multi-sensor array 200, the single sensor array 300, the processing unit 402, the sensor arrays 404a-n, the local arrays 406, the garment 500, the sensor array 502, the external network 165, the communications network 135, any device, system, and/or program in FIGS. 1-7, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 700 may include a processor 702 (e.g., a central processing unit (CPU), a graphics processing unit (GPU, or both), a main memory 704 and a static memory 706, which communicate with each other via a bus 708. The computer system 700 may further include a video display unit 710, which may be, but is not limited to, a liquid crystal display (LCD), a flat panel, a solid state display, or a cathode ray tube (CRT). The computer system 700 may include an input device 712, such as, but not limited to, a keyboard, a cursor control device 714, such as, but not limited to, a mouse, a disk drive unit 716, a signal generation device 718, such as, but not limited to, a speaker or remote control, and a network interface device 720.

The disk drive unit 716 may include a machine-readable medium 722 on which is stored one or more sets of instructions 724, such as, but not limited to, software embodying any one or more of the methodologies or functions described herein, including those methods illustrated above. The instructions 724 may also reside, completely or at least partially, within the main memory 704, the static memory 706, or within the processor 702, or a combination thereof, during execution thereof by the computer system 700. The main memory 704 and the processor 702 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 722 containing instructions 724 so that a device connected to the communications network 135, the external network 165, another network, or a combination thereof, can send or receive voice, video or data, and communicate over the communications network 135, the external network 165, another network, or a combination thereof, using the instructions. The instructions 724 may further be transmitted or received over the communications network 135, the external network 165, another network, or a combination thereof, via the network interface device 720.

While the machine-readable medium 722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a garment to be worn on a body of a user;
a flexible circuit disposed on the garment;
a sensor array disposed on the flexible circuit;
a processing unit communicatively linked to the sensor array and configured to perform operations comprising:
receiving a signal including sensor data from a sensor of the sensor array, wherein the sensor data is generated by the sensor of the sensor array while the user is at rest, while the body of the user is in motion, or a combination thereof;
processing the signal to determine a physiological condition of the user, a health condition of the user, a state of the user, or a combination thereof;
transmitting the signal, the physiological condition of the user, the health condition of the user, the state of the user, or a combination thereof, to a device for further analysis, display, storage, or a combination thereof.

2. The system of claim 1, further comprising a light emitting diode disposed on the flexible circuit and communicatively linked to the sensor array.

3. The system of claim 2, wherein the light emitting diode is configured to emit light through tissue of the body of the user.

4. The system of claim 3, wherein the sensor array is configured to measure transmission or absorption of the light in the tissue to determine a blood oxygen saturation of the user.

5. The system of claim 1, wherein the sensor array disposed on the flexible sensor array comprises a plurality of distributed sensor arrays configured to generate the sensor data.

6. The system of claim 1, further comprising a memory, a wired interface, a wireless interface, a transceiver, or a combination thereof.

7. The system of claim 1, wherein the operations further comprise receiving the signal with a plurality of other signals including additional sensor data from other sensors of the sensor array simultaneously.

8. The system of claim 1, further comprising a user interface configured to display the physiological condition of the user, the health condition of the user, the state of the user, or a combination thereof.

9. The system of claim 1, wherein the operations further comprise reducing signal noise and artifacts created while the user is in motion.

10. The system of claim 1, further comprising a sub-sensor array embedded within each sensor of the sensor array.

11. The system of claim 1, wherein the sensor data includes blood oxygen saturation data, temperature data, heart beat data, blood pressure data, motion data, ambient temperature data, relative position in space data, muscle and tissue expansion data, electrocardiogram data, respiration data, breathing rate data, sweat-related data, moisture data, fatigue data, any biometric data, any other data, or a combination thereof.

12. A method, comprising:
integrating a sensor array and a light emitting diode into a flexible circuit of a garment to be worn on a body of a user;
activating the sensor array and the light emitting diode;
obtaining sensor data from the sensor array while the user is at rest, while the user is in motion, or a combination thereof;
receiving, at a processing unit of the garment, a signal including the sensor data from the sensor array; and
processing the signal to determine a physiological condition, a health condition, a state of the user, or a combination thereof.

13. The method of claim 12, further comprising providing a power source in the garment to provide power to the sensor array and light emitting diode.

14. The method of claim 12, further comprising transmitting, after processing the signal, the signal to a device for display, further processing, storage, or a combination thereof.

15. The method of claim 12, further comprising transmitting the physiological condition, the health condition, the state of the user, or a combination thereof, to a device for display, further processing, storage, or a combination thereof.

16. The method of claim 12, further comprising facilitating continuous monitoring of the user wearing the garment by utilizing the sensor array.

17. The method of claim 12, further comprising aggregating the signal with a plurality of other signals including other sensor data at the processing unit to process and filter the signal and the other signals.

18. The method of claim 12, further comprising integrating the sensor array with a plurality of additional sensor arrays to create a distributed sensor array in the garment.

19. The method of claim 12, further comprising providing a sensor lens in each sensor of the sensor array that is configured to contact skin of the user.

20. A non-transitory computer-readable device comprising instructions, which when loaded and executed by a processor, cause the processor to perform operations comprising:
activating a sensor array and a light emitting diode integrated into a flexible circuit of a garment to be worn on a body of a user;
obtaining sensor data from the sensor array while the user is at rest, while the user is in motion, or a combination thereof;
receiving a signal including the sensor data from the sensor array; and
processing the signal to determine a physiological condition, a health condition, a state of the user, or a combination thereof.
